(19) United States
(12) Patent Application Publication
(10) Pub. No.: US 2009/0069579 A1
(43) Pub. Date: Mar. 12, 2009

(54) PROCESS AND COMPOUND

(76) Inventors: John Blacker, Huddersfield (GB); Robin Fieldhouse, New South Wales (AU)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/094,433

(22) PCT Filed: Nov. 21, 2006
(86) PCT No.: PCT/GB2006/004331

§ 371 Date: Aug. 27, 2008

(30) Foreign Application Priority Data
Nov. 21, 2005 (GB) 0523637.7

(51) Int. Cl. C07D 319/06; C07C 229/34

(52) U.S. Cl. 549/373; 560/39

(57) ABSTRACT

A process for preparing intermediate compounds useful in the preparation of statins. The process is particularly useful for the preparation of atorvastatin. The process involves the reduction of two ketone groups at the same time using either a chiral transition metal catalyst or an enzyme based system.

The present invention concerns a process and intermediate compounds useful in the preparation of statins, particularly atorvastatin.

Atorvastatin, ([R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid), was first disclosed in U.S. Pat. No. 4,681,893 which also describes its synthesis. Atorvastatin is marketed as its calcium salt under the brand name Lipitor™ and is an important drug.

Atorvastatin is a member of the drug class known as statins and is used for reducing the concentration of low density lipoprotein (LDL) in the blood stream. High concentrations of LDL have been linked to the formation of coronary lesions which obstruct the flow of blood and promote thrombosis.

The drug competitively inhibits the enzyme 3-hydroxy-3-methylglutaryl-coenzyme A reductase (HMG-COA reductase). HGG-CoA reductase catalyses the conversion of HMG to mevalonate, which is the rate determining step in the biosynthesis of cholesterol. Hence inhibition of HMG-CoA reductase leads to a decrease in the concentration of cholesterol. Decreased production of cholesterol leads to an increase in the number of LDL receptors and a corresponding reduction in the production of LDL particles by the metabolism of IDL. This has a number of beneficial therapeutic effects.

U.S. Pat. No. 5,273,995 discloses a chiral synthesis of atorvastatin. The first step involves alkylation of an aldehyde with a chiral ester fragment in the presence of MgBr₂ to form a chiral alcohol group on the ester intermediate. The ester then undergoes transesterification to the methyl ester using sodium methoxide. The methyl ester is then reacted onto a β-ketoester which is then reacted through a number of steps to yield the atorvastatin. However, the chirality in the ester group, which in effect serves as a chiral auxiliary, is lost during the synthesis and this represents a waste of chiral material. A further disadvantage of this route is the low stereoselectivity of the aldol reaction meaning that further recrystallisation steps would be necessary in order to obtain a pure diastereomeric material which would decrease the overall yield. Another disadvantage to this route is the use of the expensive, flammable and corrosive sodium methoxide during the transesterification step.

The prior art thus describes various routes to atorvastatin. However, each of the prior art processes suffers disadvantages.

In certain cases the starting materials are expensive to purchase or synthesise. In other cases there are handling concerns and there are issues of worker safety and concerning the environment. After use, spent reactants are difficult and expensive to dispose of because of the adverse effects the compounds may have on their surroundings. A further problem with the prior art processes is the fact that after convergence, further synthetic steps may be needed. Each synthetic step leads both to a reduction in yield and increases the possibility of competing side reactions. Thus the conventional reaction requires more effort to purify the final product and may not give an optimal yield.

It is an aim of the present invention to provide a synthetically efficient process for the production of novel intermediates for the preparation of atorvastatin which avoids the problems of the prior art processes. It is also an aim to provide a process in which the efficiency of convergency (i.e. the bringing together of synthetic fragments) is maximised. It is thus an aim to provide a route which offers an improved yield and/or purity relative to the existing routes. It is a further aim of the present invention to provide a process which minimizes the number of synthetic steps required and which avoids the problem of competing reactions and/or the disposal of hazardous materials and/or the need for additional work-up procedures.

We have found an improved route to the intermediate compounds which satisfy some or all of the above problems.

According to a first aspect of the present invention, there is provided a compound of formula (1), or tautomeric forms thereof:

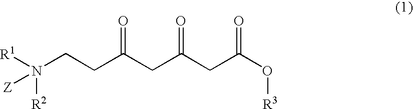

wherein
R¹ and R² each independently represents a hydrogen or a protecting group, or R¹ and R² are joined to form a cyclic protecting group;
R³ represents a hydrogen or an optionally substituted hydrocarbyl group; and
Z is hydrogen or a lone pair of electrons.

The term protecting group refers to a removable group which serves to protect the N atom during one or more synthetic steps. The term takes the meaning as defined in T W Greene “Protective Groups in Organic Synthesis” and N protecting groups defined therein are suitable for use in the present invention.

The invention specifically excludes the case where R¹ and R², together with the N atom to which they are attached form a pyrrole ring. In fact, a group of this type will not be displaceable and thus cannot serve as an N-protecting group.

In an embodiment, R³ is selected from the group comprising: H, C₁₋₇ alkyl, C₁₋₇ haloalkyl, C₁₋₇ alkylaryl, aryl C₁₋₇ alkyl, C₂₋₇ alkenyl, C₂₋₇ alkynyl, aryl, —CF₃, —CH₂F, —CHF₂, CH₂CF₃, CH₂OC₁₋₇ alkyl, CH₂OC₁₋₇ haloalkyl, CH₂SC₁₋₇ alkyl, wherein each of the above may be optionally substituted where chemically possible by 1 to 3 substituents independently selected from the group comprising: SH, OH, C₁₋₄ alkyl, CN, CF₃ and C₁₋₄ alkoxy. Preferred R³ groups are: H, C₁₋₇ alkyl, C₁₋₇ haloalkyl, aryl C₁₋₇ alkyl and C₁₋₇ alkylaryl.

In one embodiment Z is a lone pair of electrons. In an alternative embodiment Z is hydrogen. When Z is hydrogen, the compound is a quaternary ammonium compound and a counter ion is also present. Any conventional counter ion, e.g. halo is suitable.

Whilst the invention is described herein with reference to compounds with formulas as depicted, it is understood that it relates to said compounds in any possible tautomeric forms.

Hydrocarbyl groups which may be represented by R³ include alkyl, alkenyl and aryl groups, and any combination thereof, such as aralkyl and alkaryl, for example benzyl groups.

Alkyl groups which may be represented by R³ include linear and branched alkyl groups comprising up to 20 carbon atoms, particularly from 1 to 7 carbon atoms and preferably from 1 to 5 carbon atoms. When the alkyl groups are branched, the groups often comprising up to 10 branch chain carbon atoms, preferably up to 4 branch chain atoms. In certain embodiments, the alkyl group may be cyclic, commonly comprising from 3 to 10 carbon atoms in the largest ring and optionally featuring one or more bridging rings. Examples of alkyl groups which may be represented by R³ include methyl, ethyl, propyl, 2-propyl, butyl, 2-butyl, t-butyl and cyclohexyl groups.

Alkenyl groups which may be represented by R³ include C₂₋₂₀, and preferably C₂₋₆ alkenyl groups. One or more carbon—carbon double bonds may be present. The alkenyl group may carry one or more substituents, particularly phenyl substituents. Examples of alkenyl groups include vinyl, styryl, indenyl and allyl groups.

Aryl groups which may be represented by R³ may contain 1 ring or 2 or more fused rings which may include cycloalkyl, aryl or heterocyclic rings. Examples of aryl groups which may be represented by R³ include phenyl, benzyl, 2-phenethyl, tolyl, fluorophenyl, chlorophenyl, bromophenyl, trifluoromethylphenyl, anisyl, naphthyl and ferrocenyl groups.

In an embodiment, aryl includes any aromatic carbocyclic ring or ring system comprising one or more rings which may be fused, conjugated or isolated from one another and containing up to 24 carbon atoms in the ring system skeleton. Aryl thus includes systems such as phenyl, naphthyl, anthracyl, bisphenyl, phenanthryl, and indenyl.

When R³ is a substituted hydrocarbyl group, the substituent(s) should be such so as not to adversely affect the rate or selectivity of any of the reaction steps or the overall process. Optional substituents include halogen, cyano, nitro, hydroxyl, amino, thiol, acyl, hydrocarbyl, heterocyclyl, hydrocarbyloxy, mono or di-hydrocarbylamino, hydrocarbylthio, esters, carbamates, carbonates, amides, trihydrocarbylsilyl, trihydrocarbysilyloxy, sulfonyl and sulfonamide groups wherein the hydrocarbyl groups are as defined for R³ above. One or more substituents may be present. Examples of R³ groups having more than one substituent present include —CF₃ and —C₂F₅.

Protecting groups which may be represented by R¹ and R² include amine protecting groups, examples of which are well known in the art. Examples of protecting hydrocarbyloxycarbonyl groups, for example aryl- or alkyl-oxycarbonyl groups, and silyl groups, for example triaryl- and especially trialkylsilyl groups. Suitable protecting groups also include benzyloxycarbonyl and triphenylmethyl(trityl) group. Alkyl and aryl sulfonamide groups may also be used as protecting groups. Thus, C₁₋₇ alkyl, e.g. methyl, and phenyl, benzyl or tolyl sulfonamides are particularly suitable. Examples of cyclic protecting groups which may be represented by R¹ and R² include phthalimido groups. Especially preferred protecting groups or cyclic protecting groups are benzyl, acetyl, allyl, t-butyloxycarbonyl, trimethylsilyl, t-butyldimethylsilyl and t-butyldiphenylsilyl groups.

Protecting groups which may be represented by R¹ and R² may be same or different. When the protecting groups R¹ and R² are different, advantageously this may allow for the selective removal of one of only R¹ and R². Preferably, when the protecting groups R¹ and R² are different, R¹ is a benzyl and R² is an acyl or silyl group.

Preferably, R¹ is selected from benzyl, t-butyloxycarbonyl, or allyl groups; R² is selected from benzyl, t-butyloxycarbonyl or allyl groups; R³ is selected from t-butyl, methyl or ethyl groups.

More preferably, R¹ is selected from benzyl or allyl groups; R² is selected from benzyl or allyl groups; R³ is selected from t-butyl or methyl groups.

Most preferably, R¹ is a benzyl group; R² is a benzyl group; and R³ is a t-butyl group.

According to a second aspect of the present invention, there is provided a process for the preparation of a compound of formula (1):

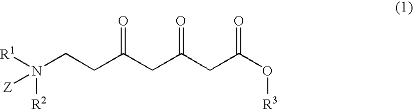

wherein
R¹ and R² each independently represents a hydrogen or a protecting group, or R¹ and R² are joined to form a cyclic protecting group;
R³ represents a hydrogen or an optionally substituted hydrocarbyl group; and
Z represents hydrogen or a lone pair of electrons,
which process comprises reacting the compound of formula (3)

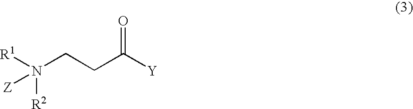

wherein
R¹ and R² each independently represents a hydrogen or a protecting group,
or R¹ and R² are joined to form a cyclic protecting group; and

### Y is OR⁴OR NR⁵R⁶

with a compound of formula (4)

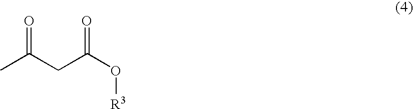

to give a compound of formula (1).

In an embodiment, the reaction is carried out in the presence of a strong base in an aprotic solvent. The base is preferably an alkali metal hydride, or an alkyl lithium compound, or a mixture of these. Other conventional strong bases can also be used. Suitable solvents include THF, diethyl ether and glymes. Other conventional aprotic solvents may also be used.

According to a third aspect of the present invention, there is provided a process for the preparation of a compound of formula (1):

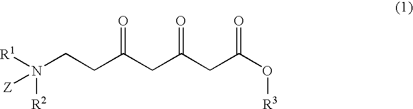

wherein
R¹ and R² each independently represents a hydrogen or a protecting group,
or R¹ and R² are joined to form a cyclic protecting group; and
R³ represents a hydrogen or an optionally substituted hydrocarbyl group, which comprises
reacting an amine of formula HNR¹R²Z
wherein
R¹ and R² each independently represents a hydrogen or a protecting group, or R¹ and R² are joined to form a cyclic protecting group, and
Z is hydrogen or a lone pair of electrons
with a compound of formula (2)

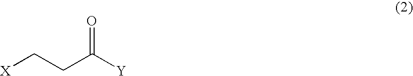

wherein
X is a leaving group; and
Y is OR⁴ or NR⁵R⁶ wherein R⁴ is an optionally substituted hydrocarbyl group, R⁵ is an optionally substituted hydrocarbyl group or an optionally substituted hydrocarbyloxy group, or R⁵ and R⁶ are joined to form a heterocyclic ring containing one or more heteroatoms, to give a compound of formula (3)

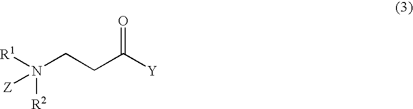

wherein
R¹ and R² each independently represents a hydrogen or a protecting group, or R¹ and R² are joined to form a cyclic protecting group;
Z is hydrogen or a lone pair of electrons; and
Y is OR⁴ or NR⁵R⁶ wherein R⁴ is an optionally substituted hydrocarbyl group, R⁵ is an optionally substituted hydrocarbyl group, R⁶ is an optionally substituted hydrocarbyl group or an optionally substituted hydrocarbyloxy group, or R⁵ and R⁶ are joined to form an optionally substituted heterocyclic ring containing one or more heteroatoms
and reacting the compound of formula (3) with a compound of formula (4)

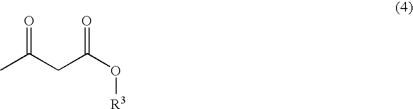

to give a compound of formula (1).

Again, when Z is hydrogen a conventional counter ion will be present to balance the charge on the N atom.

In an embodiment, the reaction of the amine (3) and diketone (4) takes place in the presence of a strong base and an aprotic solvent. Suitable strong bases are as defined previously and thus include alkali metal hydrides and alkyl lithium compounds. Other conventional strong bases can be used. The reaction preferably occurs in an aprotic solvent. THF or diethyl ether are preferred.

Optionally substituted hydrocarbyl groups which may be represented by R⁴⁻⁶ are as described above for R³. Optionally substituted hydrocarbyloxy groups which may be represented by R⁶ include alkoxy, alkenyloxy, aryloxy groups, and any combination thereof, such aralkyloxy or alkaryloxy, for example benzyloxy groups wherein the alkyl, alkenyl, aryl, alkaryl or aralkyl components are as described above for the optionally substituted hydrocarbyl group R³.

When Y is OR⁴, preferably R⁴ is a lower alkyl group, for example C₁₋₄ alkyl.

When Y is NR⁵R⁶, preferably R⁵ is a lower alkyl group, for example C₁₋₄ alkyl or C₁₋₄ alkoxy, more preferably methyl or methoxy.

When Y is NR⁵R⁶ and R⁵ and R⁶ are joined such that a heterocyclic ring containing one or more heteroatoms is formed with the nitrogen to which R⁵ and R⁶ are attached, preferably the heterocyclic ring contains from 5 to 7 rings atoms of which one or more atoms are heteroatoms selected from N, O, P or S, the remaining ring atoms being C atoms. More preferably when Y is NR⁵R⁵ and R⁵ and R⁶ are joined such that a heterocyclic ring containing one or more heteroatoms is formed with the nitrogen to which R⁵ and R⁶ are attached, the heterocyclic ring is a morpholine ring.

According to a fourth aspect of the present invention, there is provided a process for the preparation of a compound of formula (7):

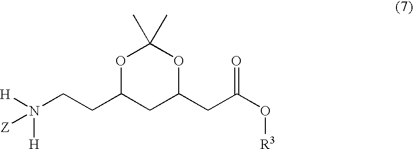

wherein
R³ represents a hydrogen or an optionally substituted hydrocarbyl group, which comprises
(a) reducing a compound of formula (1):

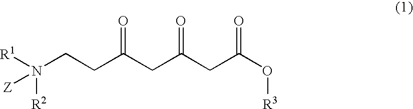

wherein
R¹ and R² each independently represents a hydrogen or a protecting group,
or R¹ and R² are joined to form a cyclic protecting group; and
R³ represents a hydrogen or an optionally substituted hyrocarbyl group, to give a compound of formula (5):

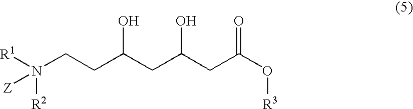

wherein
R¹ and R² each independently represents a hydrogen or a protecting group,
or R¹ and R² are joined to form a cyclic protecting group;
Z is hydrogen or a lone pair of electrons; and
R³ represents a hydrogen or an optionally substituted hydrocarbyl group,
(b) reacting the compound of formula (5) with an acetone-equivalent to give a compound of formula (6):

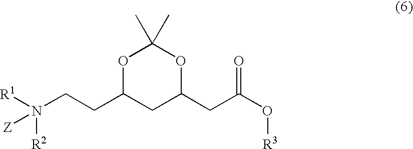

wherein
R¹ and R² each independently represents a hydrogen or a protecting group,
or R¹ and R² are joined to form a cyclic protecting group; and
R³ represents a hydrogen or an optionally substituted hydrocarbyl group, and
(c) removal of any R¹ or R² protecting groups, to give a compound of formula (7).

Reduction of compounds of formula (1) can be achieved using reduction systems known in the art for the reduction of ketone groups. Preferred reductions systems include reduction with Raney nickel and hydrogen, reduction with hydrogen in the presence of a catalyst, such as palladium on carbon, reduction using hydride reagents, such as LiAlH₄ and NaBH₄. Most preferred is reduction using boranes such as borane-THF. When palladium on carbon catalysed hydrogenation is employed, preferred conditions comprise the use of methanol solvent at elevated temperature, such as about 40° C., in the presence of from 0.01 to 100 molar equivalents of ammonia.

The reduction of compounds of formula (1) is preferably accomplished employing a stereoselective reduction system. Stereoselective reduction systems include hydrogenation using hydrogen in the presence of chiral coordination transition metal catalyst, transfer hydrogenation in the presence of chiral coordinated transition metal catalyst, chiral metal hydride systems, for example chiral borohydride reagents, and bioreductions using enzymes or whole cell systems. It is recognised that since it is necessary to reduce two keto-groups in the compound of formula (1) that this may be achieved stepwise or simultaneously and that one or more reduction systems may be employed. In a preferred embodiment, a stereoselective reduction employing a chiral coordinated transition metal catalysed transfer hydrogenation process is employed. Examples of such processes, and the catalysts, reagents and conditions employed therein include those disclosed in International patent application publication numbers WO 91/20789, WO 98/42643 and WO 02/44111 each of which is specifically incorporated herein by reference and the hydrogenation systems and catalysts described therein are specifically intended to form part of the present application.

Transfer catalysts which are particularly suitable include the following:

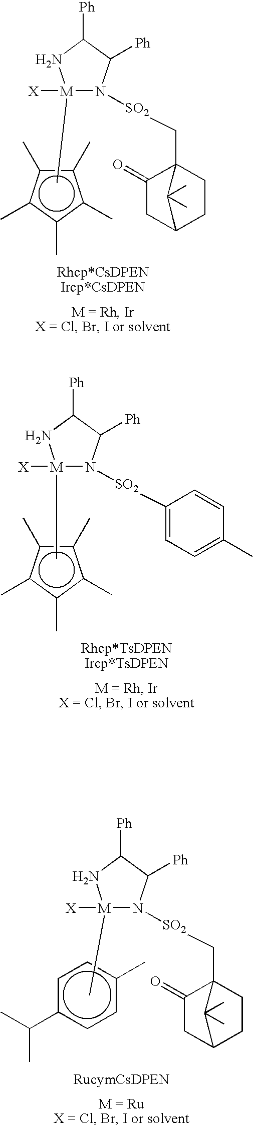

The reduction may also be accomplished using enzymatic or microbial reduction processes. Thus a reductase-supplying microorganism or a reductase obtained from such an organism could be used.

The reduction of the ketone can be carried out in a single stage or a two-stage fermentation and transformation process.

In the single stage process, the microorganisms are grown in an appropriate medium containing carbon and nitrogen sources. After sufficient growth of microorganisms, a compound of formula (1) is added to the microbial cultures and the transformation may be continued until complete conversion is obtained.

In the two-stage process, microorganisms are grown in an appropriate medium by fermentation exhibiting the desired oxido-reductase activity in the first stage. Subsequently, cells are harvested by centrifugation.

Microorganisms can be used in free state as wet cells, freeze-dried cells or heat-dried cells. Immobilized cells on support by physical adsorption or entrapment can also be used for this process. Microbially derived oxido-reductases may be used in free state or immobilized on support.

Suitable microorganisms for reduction of the ketone are Pichia methanolica ATCC 58403, Pichia pastoris ATCC 28485, Geotrichum candidum ATCC 34614, Nocardia globerula ATCC 21505 and Acinetobacter calcoaceticus ATCC 33305 or a reductase derived from any of these microorganisms.

The transformation of compound (I) may also be accomplished by reductase isolated from microorganisms. The isolation may be accomplished by homogenizing cell suspensions, followed by disintegration, centrifugation, DEAE-cellulose chromatography, Ammonium sulfate fractionation, Sephacryl chromatography, and Mono-Q chromatography.

Remaining R¹ and R² protecting groups may be removed by methods known in the art for the removal of the given protecting group. For example, silyl protecting groups may be removed by contact with a source of fluoride ion, such as tetrabutlyammonium fluoride, benzyl ethers may be removed by hydrogenolysis, BOC protecting groups may be removed by treatment with hydrazine.

Acetone equivalents include any acetone equivalents known in the art for example acetone, 2-methoxypropene or 2,2-dimethoxypropane.

According to a fifth aspect of the present invention, there is provided a process for the preparation of a compound of formula (10) or salts thereof:

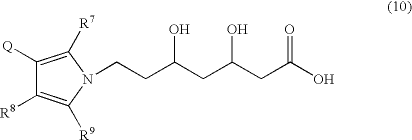

wherein
R⁷ represents a hydrogen or an optionally substituted hydrocarbyl group
R⁸ represents a hydrogen or substituent group
R⁹ represents a hydrogen or an optionally substituted hydrocarbyl group
Q represents a hydrogen or substituent group
which comprises
(a) coupling the compound of formula (7) with a compound of formula (8):

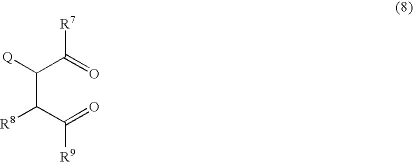

to give a compound of formula (9):

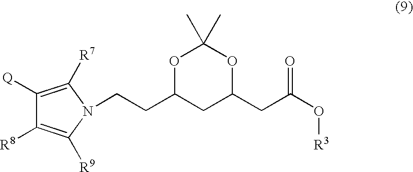

wherein
R³ represents a hydrogen or an optionally substituted hydrocarbyl group
R⁷ represents a hydrogen or an optionally substituted hydrocarbyl group
R⁸ represents a hydrogen or substituent group
R⁹ represents a hydrogen or an optionally substituted hydrocarbyl group
Q represents a hydrogen or substituent group and
(b) removing any remaining protecting groups, and hydrolysing any ester group to give a compound of formula (10) or salts thereof:

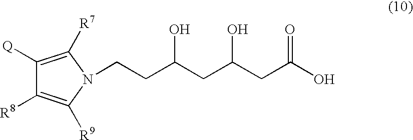

wherein the compound of formula (7) is obtained by means of any of the processes according to the second, third or fourth aspects of the present invention.

Hydrocarbyl groups which may be represented by R⁷ and R⁹ are as described for R³ and independently include alkyl, alkenyl and aryl groups, and any combination thereof, such as aralkyl and alkaryl, for example benzyl groups.

Alkyl groups which may be represented by R⁷ and R⁸ include linear and branched alkyl groups comprising up to 20 carbon atoms, particularly from 1 to 7 carbon atoms and preferably from 1 to 5 carbon atoms. When the alkyl groups are branched, the groups often comprising up to 10 branch chain carbon atoms, preferably up to 4 branch chain atoms. In certain embodiments, the alkyl group may be cyclic, commonly comprising from 3 to 10 carbon atoms in the largest ring and optionally featuring one or more bridging rings. Examples of alkyl groups which may be represented by R⁷ and R⁹ include methyl, ethyl, propyl, 2-propyl, butyl, 2-butyl, t-butyl and cyclohexyl groups.

Alkenyl groups which may be represented by R⁷ and R⁹ include C₂₋₂₀, and preferably C₂₋₆ alkenyl groups. One or more carbon-carbon double bonds may be present. The alkenyl group may carry one or more substituents, particularly phenyl substituents. Examples of alkenyl groups include vinyl, styryl and indenyl groups.

Aryl groups which may be represented by R⁷ and R⁹ may contain 1 ring or 2 or more fused rings which may include cycloalkyl, aryl or heterocyclic rings. Examples of aryl groups which may be represented by R¹ and R² include phenyl, tolyl, fluorophenyl, chlorophenyl, bromophenyl, trifluoromethylphenyl, anisyl, naphthyl and ferrocenyl groups.

When any of R⁷ and R⁹ is a substituted hydrocarbyl group, the substituent(s) should be such so as not to adversely affect the rate or selectivity of any of the reaction steps or the overall process. Optional substituents include halogen, cyano, nitro, hydroxyl, amino, thiol, acyl, hydrocarbyl, heterocyclyl, hyrocarbyloxy, mono or di-hydrocarbylamino, hydrocarbylthio, esters, carbamates, carbonates, amides, sulfonyl and sulfonamide groups wherein the hydrocarbyl groups are as defined for R⁷ above. One or more substituents may be present. Examples of R⁷ or R⁹ groups having more than one substituent present include —CF₃ and —C₂F₅.

Substituent groups which may be represented by Q and R⁸ independently include hydrocarbyl groups as defined above for R⁷, electron donating groups, electron withdrawing groups, halogens and heterocyclic groups. Substituent groups are commonly selected from the group consisting of optionally substituted alkoxy (preferably C₁₋₄ alkoxy), optionally substituted aryl (preferably phenyl), optionally substituted aryloxy (preferably phenoxy), polyalkylene oxide (preferably polyethylene oxide or polypropylene oxide), carboxy, phosphato, sulfo, nitro, cyano, halo, ureido, —SO₂F, hydroxyl, ester, —NR^{a}R^{b}, —COR^{a}, —CONR^{a}R^{b}, —NHCOR^{a}, —OCONR^{a}R^{b}, carboxyester, sulfone, and —SO₂NR^{a}R^{b} wherein R^{a} and R^{b} are each independently H, optionally substituted aryl, especially phenyl, or optionally substituted alkyl (especially C₁₋₄ alkyl) or, in the case of —NR^{a}R^{b}, —CONR^{a}R^{b} and —SO₂NR^{a}R^{b}, R^{a} and R^{b} may also together with the nitrogen atom to which they are attached represent an aliphatic or aromatic ring system; or a combination thereof.

Preferably, there is provided a process for the preparation of a compound of formula (10) or salts thereof:

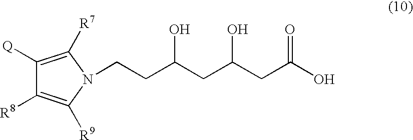

wherein
R⁷ represents an optionally substituted alkyl group, such as a C₁₋₆ alkyl group, and preferably and isopropyl group
R⁸ represents an optionally substituted aryl group, preferably a phenyl group
R⁹ represents an optionally substituted aryl group, preferably a 4-fluorophenyl group
Q represents a group of formula —COW, wherein W represents —OR¹⁰, in which R¹⁰ represents an optionally substituted alkyl, preferably a methyl or ethyl group or —NR¹¹R¹², wherein R¹¹ and R¹² each independently represent H, an optionally substituted alkyl, or an optionally substituted alkyl, or an optionally substituted aryl, and preferably R¹¹ is H and R¹² is phenyl which comprises
(a) coupling the compound of formula (7) with a compound of formula (8):

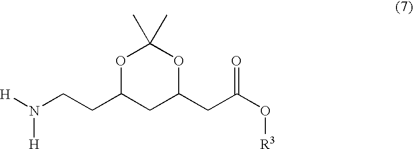

to give a compound of formula (9):

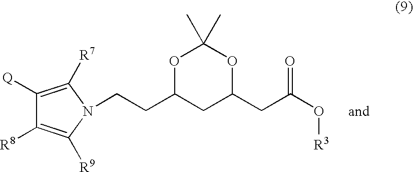

(b) removing any remaining protecting groups, and hydrolysing any ester group to give a compound of formula (10) or salts thereof:

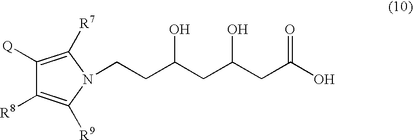

The optional substituents in groups R⁷ to R¹² include those defined for R³ and are chosen independently.

More preferably R⁷ is an isopropyl group, R⁸ is a phenyl group, R⁹ is a 4-fluorphenyl group and Q is a —CO₂Me, —CO₂Et or —CONHPh group.

The coupling of the compound of formula (7) with the compound of formula (8) may employ conditions analogous to those given in WO 89/07598 for the corresponding coupling. The conditions preferably comprise refluxing the compounds of formula (7) and (8) in a hydrocarbon solvent, such as toluene or cyclohexane, or mixtures thereof, followed by contact with aqueous acid, such as aqueous HCl.

Remaining protecting groups may be removed by methods known in the art for the removal of the given protecting group. For example, silyl protecting groups may be removed by contact with a source of fluoride ion, such as tetrabutylammonium fluoride, benzyl ethers may be removed by hydrogenolysis, and acetals and ketals may be removed by treatment with dilute aqueous acid.

It will be recognised that when Q represents a group of formula —COOR¹⁰, this may be converted to a group wherein Q represents —CONR¹¹R¹² with a compound of formula HNR¹¹R¹².

The skilled man will appreciate that the compounds of the invention could be made by adaptation of the methods herein described and/or adaptation of methods known in the art. Such modification to the processes of the present invention are also intended to form part of the invention. Thus the skilled person could modify the claimed processes by reference to standard textbooks such as “Comprehensive Organic Transformations—A Guide to Functional Group Transformations”, R C Larock, Wiley-VCH (1999 or later editions), “March's Advanced Organic Chemistry, Part B, Reactions and Synthesis”, F A Carey, R J Sundberg, Kluwer Academic/Plenum Publications, (2001 or later editions), “Organic Synthesis—The Disconnection Approach”, S Warren (Wiley), (1982 or later editions), “Designing Organic Syntheses” S Warren (Wiley) (1983 or later editions), “Guidebook to Organic Synthesis” R K Mackie and D M Smith (Longman) (1982 or later editions), etc, and the references therein as a guide.

It will be apparent to those skilled in the art that sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and references therein.

One advantage of the process of the present invention is that a double reduction of the two ketone groups that eventually become chiral alcohol groups in the product are reduced at the same time. In the prior art, the two groups are normally reduced in separate steps. There has been no reported double reduction of the keto-amine fragment regardless of whether the amine is in protected or unprotected form. The process of the present invention thus represents a major advantage in terms of reducing the cost and complexity of the overall synthesis to atorvastatin. The present invention allows a protected, or unprotected in the case of certain enzymes, amine diketone to be reduced to the corresponding di-alcohol compound in a single step. The yields are good. There is also good control of the stereochemistry. Both of these factors represent significant advantages.

Compounds of formula (10) are advantageously converted to pharmaceutically acceptable salts, especially their calcium salts.

Preferred compounds of formula (5) are compounds of formula:

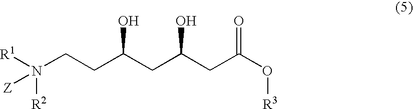

wherein R¹, R² and R³ are as previously described.

Preferred compounds of formula (6) are compounds of formula:

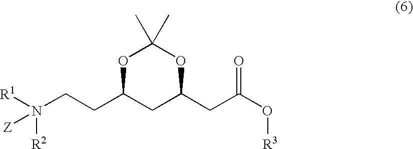

wherein R¹, R² and R³ are as previously described.

Preferred compounds of formula (7) are compounds of formula:

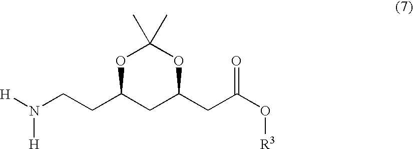

wherein R³ is as previously described.

Preferred compounds of formula (9) are compounds of formula:

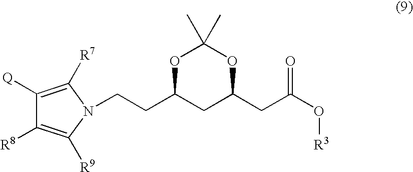

wherein R³, R⁷, R⁸, R⁹ and Q are as previously described.

Preferred compounds of formula (10) are compounds of formula:

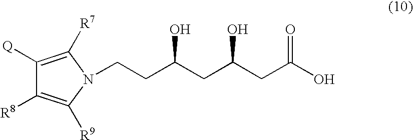

wherein R⁷, R⁸, R⁹ and Q are as previously described.

Compounds of formula (8) are advantageously prepared by the methods given in J. Med. Chem., 1991, 34, pp 357-366. Particularly preferred compounds of formula (8) are compounds of formula:

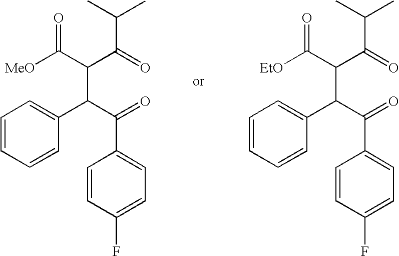

The invention is illustrated by the following examples.

### Step (1)˜Formation of the Dibenzylamino Ester

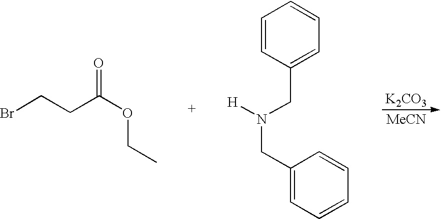

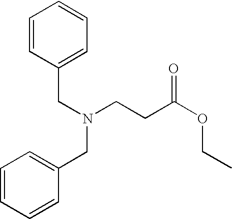

| | | | | Number | Mol |
|---|---|---|---|---|---|
| Material | M Wt | Str | Actual | of Moles | Ratio |
| Ethyl-3-bromopropanoate | 181.03 | 99% | 2.7310 g | 0.15 | 1 |
| Dibenzylamine | 197.28 | 97% | 2.9587 g | 0.15 | 1 |
| Anhydrous | 140 | — | 8.4718 g | 0.60 | 4 |
| K₂CO₃ | | | | | |
| Anhydrous MeCN | 41.05 | — | 50 cm³ | — | — |

In a round bottom flask, to a mixture of the 3-bromopropyl ester and anhydrous K₂CO₃ in anhydrous MeCN, the dibenzylamine was added. The mixture was stirred under reflux for 3 hours. The mixture was then allowed to cool to room temperature and then filtered through Celite. The solution was then concentrated under reduced pressure, and purified using column chromatography (80% Hexane/20% Ethyl acetate). The yield was ca. 10%.

TLC: R_{f}=0.54 (80120 Hexane/Ethyl acetate)

GMS: 297 [M⁺], 252 [(M-OCH₂CH₃)⁺], 210 [(M-CH₂CO₂CH₂CH₃)⁺], 206 [(M-PhCH₂)⁺], 91 [PhCH₂⁺].

¹H NMR (300 MHz, CDCl₃)-1.13 (t, 3H, J=7.1, CH₃), 2.42 (t, 2H, J=7.2, NCH₂CH₂), 2.74 (T, 2H, J=7.2, CH₂CO), 3.51 (s, 4H, N(CH₂Ph)₂), 4.01 (q, 2H, J=7.1, CO₂CH₂CH₃), 7.02 (M, 10H ArH).

### Step (2)—Formation of the DibenzylAminoDiketone

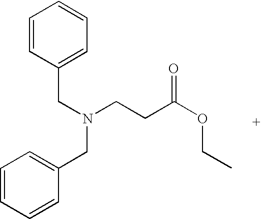

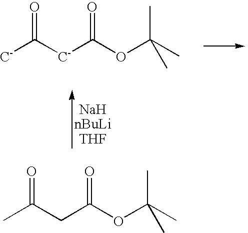

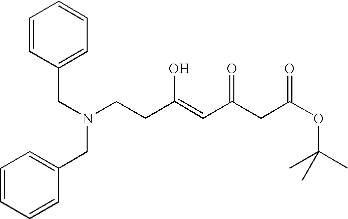

| | | | | Number | Mole |
|---|---|---|---|---|---|
| Material | Str | Mwt | Actual | of Moles | Ratio |
| NaH | 60% | 28 | 0.0216 | 4.627 × 10⁻⁴ | 1 |
| Anhydrous | 99% | 72.11 | 7 cm³ | — | — |
| THF | | | | | |
| ⁿBuLi | 2.5 M (in | 64.06 | 0.23 cm³ | 5.090 × 10⁻⁴ | 1.1 |
| | Hexane) | | | | |
| ^{t}Butyl | 99% | 158 | 0.0731 g | 4.627 × 10⁻⁴ | 1 |
| acetoacetate | | | | | |
| Dibenzyl- | — | 297.40 | 0.1376 g | 4.627 × 10⁻⁴ | 1 |
| AminoEster | | | | | |
| pH 10 Buffer | — | — | 20 cm³ | — | — |
| Ethyl acetate | — | — | 30 cm³ | — | — |
| Brine | — | — | 20 cm³ | — | — |
| NaHCO₃ | — | — | 10 cm³ | — | — |
| MgSO₄ | — | — | — | — | — |

To a round bottom flask under nitrogen was added NaH along with anhydrous THF. With stirring the ^{t}Butyl acetoacetate was added. The mixture was then cooled to 0° C. using an ice bath. The nBuLi was then added, maintaining the temperature at 0° C., and the solution was then left to stir at 0° C. for 10 mins. The solution was then cooled to −78° C., the DibenzylAminoEster was charged, and the mixture left to stir for a further 45 mins. The temperature was then increased to −30° C. over a period of 30 mins, held at −30° C. for 15 mins, then cooled back down to −78° C. After a further 15 mins at −78° C. the solution was quenched into an ice cooled solution of the pH 10 buffer and ethyl acetate. The layers were separated and the aqueous washed 3 times with 10 cm³ of ethyl acetate. The combined organic phase was washed with 20 cm³ of a 5% NaHCO₃ solution, 20 cm³ of water, and then with 20 cm³ of Brine. The resulting organic phase was dried using MgSO₄ and concentrated under reduced pressure. The product was purified using column chromatography (80% Hexane/20% Ethyl acetate). The yield of 2b was ˜30%.

TLC: Rf=0.33 (80/20 Hexane/Ethyl acetate)

¹H NMR (300 MHz, CDCl₃)-1.46 (S, 9H, C(CH₃)₃, 2.47 (t, 2H, J=7.4 CH₂CO), 2.78 (t, 2H, J=7.4 NCH₂CH₂), 3.19 (s, 2H, COCH₂CO₂Et), 3.58 (s, 4H, N(CH₂Ph)₂), 5.46 (s, 1H, vinylic H), 7.31 (m, 10H, ArH).

1. A compound of formula (1), or tautomeric forms thereof; 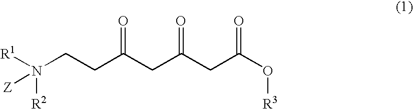
wherein
R¹ and R² each independently represents a hydrogen or a protecting group,
or R¹ and R² are joined to form a cyclic protecting group;
R³ represents a hydrogen or an optionally substituted hydrogen or an optionally substituted hydrocarbyl group; and
Z represents hydrogen or a lone pair of electrons.

2. A process for the preparation of a compound of formula (1). 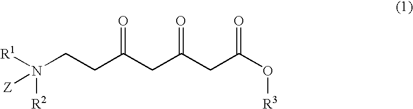
wherein
R¹ and R² each independently represents a hydrogen or a protecting group,
or R¹ and R² are joined to form a cyclic protecting group;
R³ represents a hydrogen or an optionally substituted hydrocarbyl group; and
Z represents hydrogen or a lone pair of electrons,
which process comprises reacting the compound of formula (3) 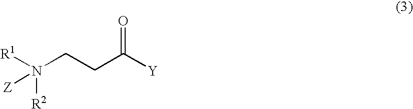
wherein
R¹ and R² each independently represents a hydrogen or a protecting group,
or R¹ and R² are joined to form a cyclic protecting group;
Z represents hydrogen or a lone pair of electrons; and
Y is OR⁴ OR NR⁵R⁶
with a compound of formula (4) 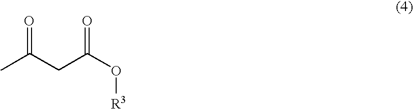
to give a compound of formula (1).

3. A process for the preparation of a compound of formula (1): 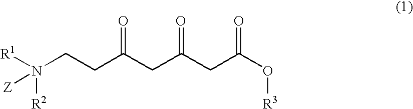
wherein
R¹ and R² each independently represents a hydrogen or a protecting group,
or R¹ and R² are joined to form a cyclic protecting group;
R³ represents a hydrogen or all optionally substituted hydrocarbyl group; and
Z represents hydrogen or a lone pair of electrons,
which process comprises
(a) reacting an amine of formula HNR¹R²Z
wherein
R¹ and R² each independently represent a hydrogen or a protecting group, or R¹ add
R² are joined to form a cyclic protecting group; and
Z is hydrogen or a one pair,
with a compound of formula (2) 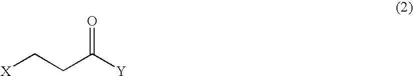
wherein
X is a leaving group; and
Y is OR⁴ or NR⁵R⁶ wherein R⁴ is au optionally substituted hydrocarbyl group, R⁵ is an optionally substituted hydrocarbyl group, R⁶ is an optionally substituted hydrocarbyl soup or an optionally substituted hydrocarbyloxy group, or R⁵ and R⁶ are joined to form a heterocyclic ring containing one or more heteroatoms,
to give a compound of formula (3) 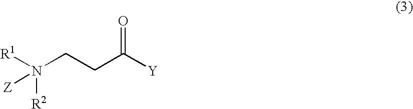
wherein
R¹ and R² each independently represents a hydrogen or a protecting group,
or R¹ and R² are joined to form a cyclic protecting group; and
Y is OR⁴ or NR⁵R⁶ wherein R⁴ is an optionally substituted hydrocarbyl group, R⁵ is an optionally substituted hydrocarbyl group, R⁶ is an optionally substituted hydrocarbyl group or an optionally substituted hydrocarbyloxy group, or R⁵ and R⁶ are joined to form an optionally substituted heterocyclic ring containing one or more heteroatoms; and
Z represents hydrogen or a lone pair of electrons, and
(a) reacting the compound of formula (3) with a compound of formula (4) 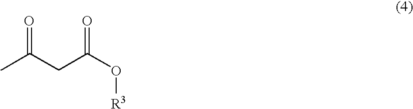
to give a compound of formula (1).

4. A process for the preparation of a compound of formula (7): 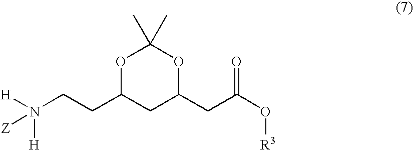
wherein
R³ represents a hydrogen or an optionally substituted hydrocarbyl group; and
Z represents hydrogen or a lone pair of electrons;
which process comprises
a) reducing a compound of formula (1): 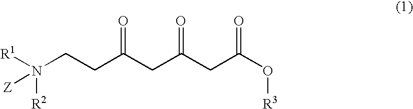
wherein
R¹ and R² each independently represents a hydrogen or a protecting group,
or R¹ and R² are joined to form a cyclic protecting group;
R³ represents a hydrogen or an optionally substituted hydrocarbyl group; and
Z represents hydrogen or a lone pair of electrons,
to give a compound of formula (5): 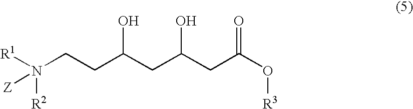
wherein
R¹ and R² each independently represents a hydrogen or a protecting group,
or R¹ and R² are joined to form a cyclic protecting group;
R³ represents a hydrogen or an optionally substituted hydrocarbyl group; ad
Z represents hydrogen or a lone pair of electrons,
(b) reacting the compound of formula (5) with an acetone-equivalent to give a compound of formula (6): 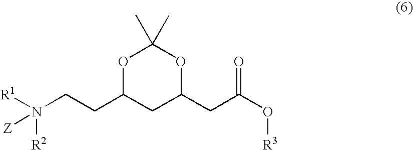
wherein
R¹ and R² each independently represents a hydrogen or a protecting group,
or R¹ and R² are joined to for a cyclic protecting group;
R³ represents a hydrogen or an optionally substituted hydrocarbyl group; and
Z represents hydrogen or a lone pair of electrons, and
(b) removal of any R¹ or R² preventing soups, to give a compound of formula (7).
